(19) United States
(12) Patent Application Publication
(10) Pub. No.: US 2016/0016969 A1
(43) Pub. Date: Jan. 21, 2016

(54) ISOHEXIDE MONOTRIFLATES AND PROCESS FOR SYNTHESIS THEREOF

(71) Applicant: ARCHER DANIELS MIDLAND COMPANY, Decatur, IL (US)

(72) Inventor: Kenneth Stensrud, Decatur, IL (US)

(73) Assignee: Archer Daniels Midland Company, Decatur, IL (US)

(21) Appl. No.: 14/771,818

(22) PCT Filed: Feb. 18, 2014
(86) PCT No.: PCT/US14/16758

§ 371 Date: Sep. 1, 2015

Related U.S. Application Data
(60) Provisional application No. 61/772,637, filed on Mar. 5, 2013.

(51) Int. Cl. C07D 493/04; C07D 519/00

(52) U.S. Cl. CPC C07D 493/04; C07D 519/00

(57) ABSTRACT

Isohexide monotriflate compounds and a method of preparing the same are described. The method involves reacting a mixture of an isohexide, a trifluoromethanesulfonate anhydride, and either 1) a nucleophilic base or 2) a combination of a non-nucleophilic base and a nucleophile. The isohexide monotriflate compounds can serve as precursor materials from which various derivative compounds can be synthesized.

### PRIORITY CLAIM

The present application claims benefit of priority of U.S. Provisional Application No. 61/772,637, filed on Mar. 5, 2013, the contents of which are incorporated herein.

### FIELD OF INVENTION

The present invention relates to cyclic bi-functional mono-trifluoromethanesulfonic acid (triflate) monomers derived from renewable materials, to particular methods by which such monomers are made, and to derivative compounds or materials incorporating these monomers.

### BACKGROUND

Traditionally, polymers and commodity chemicals have been prepared from petroleum-derived feedstock. As petroleum supplies have become increasingly costly and difficult to access, interest and research has increased to develop renewable or “green” alternative materials from biologically-derived sources for chemicals that will serve as commercially acceptable alternatives to conventional, petroleum-based or -derived counterparts, or for producing the same materials as produced from fossil, non-renewable sources.

One of the most abundant kinds of biologically-derived or renewable alternative feedstock for such materials is carbohydrates. Carbohydrates, however, are generally unsuited to current high temperature industrial processes. Compared to petroleum-based, hydrophobic aliphatic or aromatic feedstocks with a low degree of functionalization, carbohydrates such as polysaccharides are complex, over-functionalized hydrophilic materials. As a consequence, researchers have sought to produce biologically-based chemicals that can be derived from carbohydrates, but which are less highly functionalized, including more stable bi-functional compounds, such as 2,5-furandicarboxylic acid (FDCA), levulinic acid, and 1,4:3,6-dianhydrohexitols.

1,4:3,6-Dianhydrohexitols (also referred to herein as isohexides) are derived from renewable resources from cereal-based polysaccharides. Isohexides embody a class of bicyclic furanodiols that derive from the corresponding reduced sugar alcohols (D-sorbitol, D-mannitol, and D-iditol respectively). Depending on the chirality, three isomers of the isohexides exist, namely: A) isosorbide, B) isomannide, and C) isoidide, respectively; the structures of which are illustrated in Scheme 1.

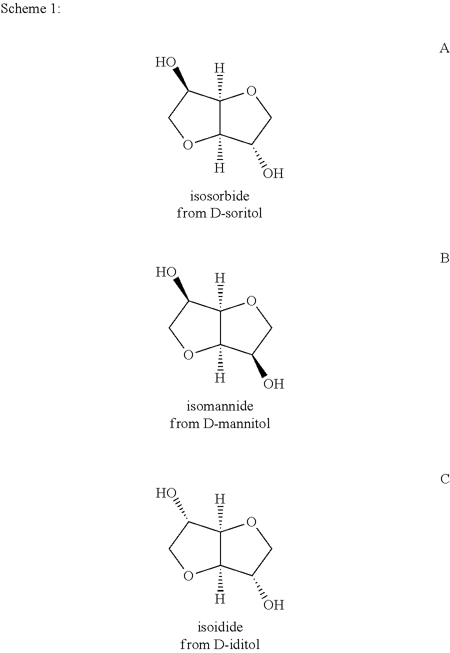 [SMILES: O[C@@H]1CO[C@H]2[C@@H]1OC[C@@H]2O ; O[C@@H]1CO[C@H]2[C@@H]1OC[C@H]2O ; O[C@H]1CO[C@H]2[C@@H]1OC[C@@H]2O]

These molecular entities have received considerable interest and are recognized as valuable, organic chemical scaffolds for a variety of reasons. Some beneficial attributes include relative facility of their preparation and purification, the inherent economy of the parent feedstocks used, owing not only to their renewable biomass origins, which affords great potential as surrogates for non-renewable petrochemicals, but perhaps most significantly the intrinsic chiral bi-functionalities that permit a virtually limitless expansion of derivatives to be designed and synthesized.

The isohexides are composed of two cis-fused tetrahydrofuran rings, nearly planar and V-shaped with a 120° angle between rings. The hydroxyl groups are situated at carbons 2 and 5 and positioned on either inside or outside the V-shaped molecule. They are designated, respectively, as endo or exo. Isoidide has two exo hydroxyl groups, while the hydroxyl groups are both endo in isomannide, and one exo and one endo hydroxyl group in isosorbide. The presence of the exo substituents increases the stability of the cycle to which it is attached. Also exo and endo groups exhibit different reactivities since they are more or less accessible depending on the steric requirements of the derivatizing reaction.

As interest in chemicals derived from natural resources is increases, potential industrial applications have generated interest in the production and use of isohexides. For instance, in the field of polymeric materials, the industrial applications have included use of these diols to synthesize or modify polycondensates. Their attractive features as monomers are linked to their rigidity, chirality, non-toxicity and the fact that they are not derived from petroleum. For these reasons, the synthesis of high glass transition temperature polymers with good thermo-mechanical resistance and/or with special optical properties is possible. Also the innocuous character of the molecules opens the possibility of applications in packaging or medical devices. For instance, production of isosorbide at the industrial scale with a purity satisfying the requirements for polymer synthesis suggests that isosorbide can soon emerge in industrial polymer applications. (See e.g., F. Fenouillot et al., “Polymers From Renewable 1,4:3,6-Dianhydrohexitols (Isosorbide, Isommanide and Isoidide): A Review,” PROGRESS IN POLYMER SCIENCE, vol. 35, pp. 578-622 (2010), or X. Feng et al., “Sugar-based Chemicals for Environmentally sustainable Applications,” CONTEMPORARY SCIENCE OF POLYMERIC MATERIALS, Am. Chem. Society, December 2010, contents of which are incorporated herein by reference.)

To better take advantage of isohexides as a green feedstock, a clean and simple method of preparing the isohexides as a platform chemical or precursor that can be subsequently modified to synthesize other compounds would be welcome by those in the green or renewable chemicals industry.

### SUMMARY OF THE INVENTION

The present invention pertains, in-part, to a process for preparing isohexide monotriflate compounds. The method involves reacting a mixture of an isohexide, a trifluoromethanesulfonate anhydride, and reagent of either 1) a nucleophilic base or 2) a combination of a non-nucleophilic base and a nucleophile.

Further, the present invention relates to the isohexide monotriflate compounds made according to the process described herein and the use thereof as platform chemicals for subsequent modification or derivatization into other chemical compounds. In particular, the monotriflates include:
a) (3R,3aS,6S,6aR)-6-hydroxyhexahydrofuro[3,2-b]furan-3-yl trifluoromethanesulfonate;
b) (3S,3aS,6R,6aR)-6-hydroxyhexahydrofuro[3,2-b]furan-3-yl trifluoromethanesulfonate;
c) (3R,3aS,6R,6aR)-6-hydroxyhexahydrofuro[3,2-b]furan-3-yl trifluoromethanesulfonate;
d) (3S,3aS,6S,6aR)-6-hydroxyhexahydrofuro[3,2-b]furan-3-yl trifluoromethanesulfonate;
e) (3R,3aS,6aR)-2,3,3a,6a-tetrahydrofuro[3,2-b]furan-3-yl trifluoromethanesulfonate;
f) (3S,3aS,6aR)-2,3,3a,6a-tetrahydrofuro[3,2-b]furan-3-yl trifluoromethanesulfonate.
   These monotriflates of isosorbide, isomannide and isoidide, respectively, are compounds that have desirable properties or characteristics for new polymer, surfactant, plasticizer, or other derivatized products.

In other aspects, the present invention relates to a process for making certain derivative compounds of an isohexide monotriflate, and the derivative compounds that are synthesized through further reactions, such as esterification, etherification, polymerization, thiolation, or amination, etc., which modify the isohexide monotrifate. The derivative compounds can include: amines, monocarboxylic acids, amphiphiles, thiols/thiol-ethers, and some polymers. A derivative compound has a general formula of either: X—R or R₁—X—R₂, wherein said X is an isohexide monotriflate, and R, R₁, R₂ each is an organic moiety that contains at least one of the following: an amine, amide, carboxylic acid, cyanide, ester, ether, thiol, alkane, alkene, alkyne, cyclic, aromatic, or a nucleophilic moiety.

### DETAILED DESCRIPTION OF THE INVENTION

### Section I.—Description

As biomass derived compounds that afford great potential as surrogates for non-renewable petrochemicals, 1,4:3,6-dianhydrohexitols are a class of bicyclic furanodiols that are valued as renewable molecular entities. (For sake of convenience, 1,4:3,6-dianhydrohexitols will be referred to as “isohexides” in the Description hereinafter.) As referred to above, the isohexides are good chemical platforms that have recently received interest because of their intrinsic chiral bi-functionalities, which can permit a significant expansion of both existing and new derivative compounds that can be synthesized.

Isohexide starting materials can be obtained by known methods of making respectively isosorbide, isomannide, or isoidide. Isosorbide and isomannide can be derived from the dehydration of the corresponding sugar alcohols, D-sorbitol and D mannitol. As a commercial product, isosorbide is also available easily from a manufacturer. The third isomer, isoidide, can be produced from L-idose, which rarely exists in nature and cannot be extracted from vegetal biomass. For this reason, researchers have been actively exploring different synthesis methodologies for isoidide. For example, the isoidide starting material can be prepared by epimerization from isosorbide. In L. W. Wright, J. D. Brandner, J. Org. Chem., 1964, 29 (10), pp. 2979-2982, epimerization is induced by means of Ni catalysis, using nickel supported on diatomaceous earth. The reaction is conducted under relatively severe conditions, such as a temperature of 220° C. to 240° C. at a pressure of 150 atmosphere. The reaction reaches a steady state after about two hours, with an equilibrium mixture containing isoidide (57-60%), isosorbide (30-36%) and isomannide (5-7-8%). Comparable results were obtained when starting from isoidide or isomannide. Increasing the pH to 10-11 was found to have an accelerating effect as well as increasing the temperature and nickel catalyst concentration. A similar disclosure can be found in U.S. Pat. No. 3,023,223, which proposes to isomerize isosorbide or isomannide. More recently, P. Fuertes proposed a method for obtaining L-iditol (precursor for isoidide), by chromatographic fractionation of mixtures of L-iditol and L-sorbose (U.S. Patent Publication No. 2006/0096588; U.S. Pat. No. 7,674,381 B2). L-iditol is prepared starting from sorbitol. In a first step sorbitol is converted by fermentation into L-sorbose, which is subsequently hydrogenated into a mixture of D-sorbitol and L-iditol. This mixture is then converted into a mixture of L-iditol and L-sorbose. After separation from the L-sorbose, the L-iditol can be converted into isoidide. Thus, sorbitol is converted into isoidide in a four-step reaction, in a yield of about 50%. (The contents of the cited references are incorporated herein by reference.)

Trifluoromethanesulfonate, also known by the name triflate, is a functional group with the formula CF₃SO₃-, and is commonly denoted as -OTf. A triflic anhydride is a compound with a formula (CF3SO₂)₂O formed of two triflate moieties. Excluding molecular nitrogen, the triflate moiety is one of the best nucleofuges (i.e., leaving groups) in the realm of organic synthesis, permitting both elimination and nucleophilic substitution events to be facilely rendered through tight control of reaction conditions, such as temperature, solvent, and stoichiometry.

### A.—Preparation of Isohexide Monotriflates

The present invention provides, in part, an efficient and facile process for synthesizing isohexide mono-trifluoromethanesulfonates (i.e., monotriflates). The process involves the reaction of an isohexide, a trifluoromethanesulfonate anhydride, and a reagent of either 1) a nucleophilic base or 2) a combination of a non-nucleophilic base and a nucleophilic, as two separate reagents species. These two reaction pathways are illustrated in Schema 2 and 4, respectively. Isohexide monotriflates are useful precursor chemical compounds for a variety of potential products, including for instance, polymers, chiral auxiliaries (e.g., for asymmetic synthesis used in pharmaceutical production), surfactants, or solvents. The present synthesis process can result in copacetic yields of corresponding mono-sulfonate, as demonstrated in the accompanying examples. The process is able to produce primarily isohexide mono-triflates in reasonably high molar yields of at least 50% from the isohexide starting materials, typically about 55%-70%. With proper control of the reaction conditions and time, one can achieve a yield of about 80%-90% or better of the monotriflate species. The isohexide is at least one of the following: isosorbide, isomannide, and isoidide. The respective isohexide compounds can be obtained either commercially or synthesized from relatively inexpensive, widely-available biologically-derived feedstocks.

According to a first embodiment or pathway, the process involves reacting initially a nucleophilic base with trifluoromethanesulfonate anhydride to generate a reactive intermediate, then adding an isohexide to the reaction to generate the isohexide triflate, such as presented in Scheme 2.

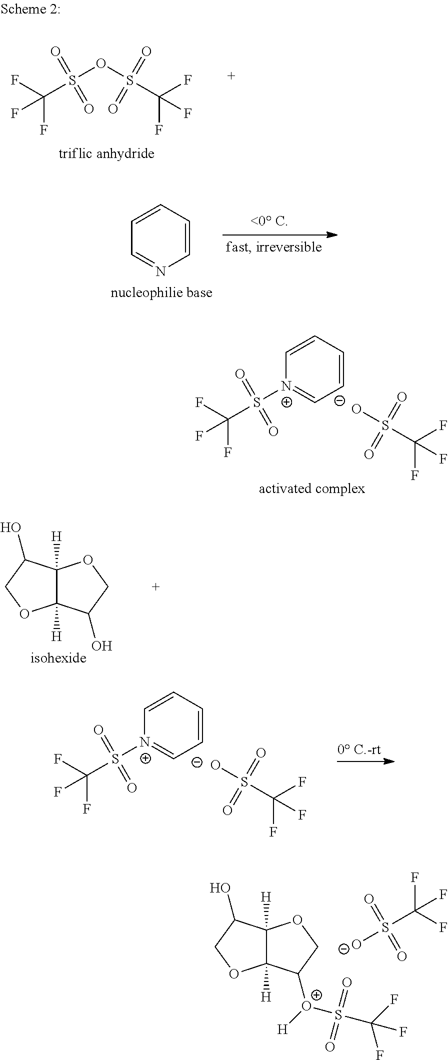

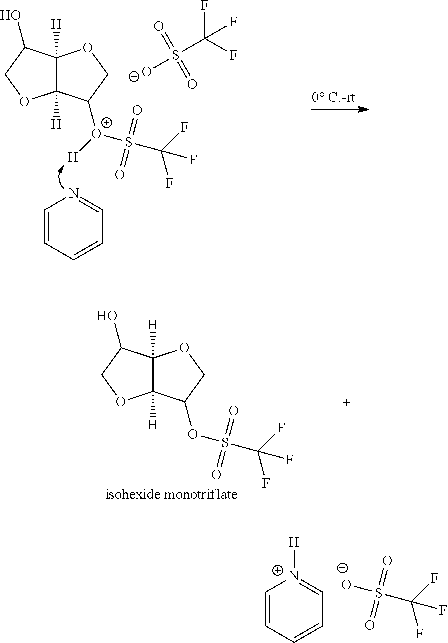 [SMILES: O=S(=O)([OH+]C1CO[C@@H]2C(O)CO[C@H]12)C(F)(F)F ; O=S(=O)([O-])C(F)(F)F ; c1ccncc1 ; O=S(=O)(OC1CO[C@@H]2C(O)CO[C@H]12)C(F)(F)F ; c1cc[nH+]cc1 ; O=S(=O)([O-])C(F)(F)F]

This reaction exhibits a relatively fast kinetics and generates an activated triflic complex. This reaction is essentially irreversible, as the liberated triflate is entirely non-nucleophilic. The triflic complex then reacts readily with the isohexide, forming an isohexide monotriflate with concomitant release and protonation of the nucleophilic base.

The single reactive species is both a nucleophile and a base that can deprotonate the hydroxyl-group of the isohexide anhydride. Different reagents can be employed as a nucleophilic base in the present synthesis process. Some common nucleophilic bases that can be used may include, for example: pyridine, derivative thereof, or structurally similar entity, such as dimethyl-aminopyridine, imidazole, pyrrolidine, and morpholine. In particular embodiments, pyridine is favored because of its inherent nucleophilic and alkaline attributes, relative low cost, and ease of removal (e.g., evaporation, water solubility, filtration (protonated form) from solution.

In certain protocols, the synthesis process involves reacting the trifluoromethanesulfonic anhydride with the nucleophilic base prior to an addition of the isohexide so as to activate the anhydride and form a labile, ammonium (e.g., pyridinium) intermediate (Scheme 3), which it is believed enables the poorly nucleophilic alcohol(s) of the isohexide to directly substitute, forming the isohexide monotriflate compound and to both release and protonate the nucleophilic base.

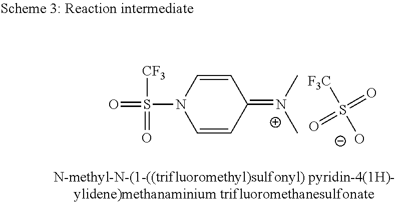 [SMILES: C[N+](C)=c1ccn(S(=O)(=O)C(F)(F)F)cc1 ; O=S(=O)([O-])C(F)(F)F]

As a second-order reaction, the reaction is conducted at a relatively low initial temperature, which permits one to control the reaction kinetics to produce a single desired compound and helps minimize the generation of a mixture of different byproducts in significant amounts. In other words, the cool to cold initial temperature helps lower the initial energy of the system, which increases control of the kinetics of the reaction, so that one can produce selectively more of the monotriflate species than of the ditriflate species. The reaction is conducted preferably at an initial temperature of about 1° C. or less. In certain embodiments, the initial temperature is typically in a range between about 0° C. or about −5° C. and about −78° C. or −80° C. In some embodiments, the initial temperature can range between about −2° C. or −3° C. and about −60° C. or −75° C. (e.g., −10° C., −15° C., −25° C., or −65° C.). Particular temperatures can be from about −5° C. or −7° C. to about −45° C. or −55° C. (e.g., −12° C., −20° C., −28° C., or −36° C.).

As the synthesis reaction uses an excess amount of a nucleophilic base, any acid that may be formed in the reaction (e.g., protonated form of isosorbide) immediately will be deprotonated, hence the pH will be alkaline (i.e., greater than 7).

In a second embodiment or pathway, as shown in Scheme 4, triflic anhydride is reacted directly with an isohexide.

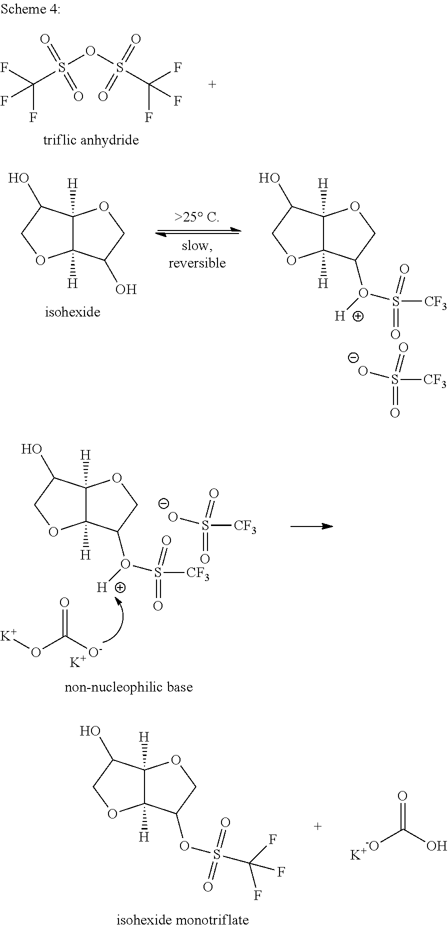

This reaction is reversible and exhibits relatively slow kinetics; hence, heat is added to help promote formation of the intermediate and drive the reaction. A non-nucleophilic base, such as potassium carbonate, is employed to deprotonate the monotriflate isohexide compound. Some common non-nucleophilic bases that may be employed in the reaction include, for example: carbonates, bicarbonates, acetates, or anilines. This reaction is usually performed at about ambient room temperatures (20° C.-25° C.) or greater. In some reactions, the temperature can be as high as about 130° C. or 140° C., but typically is about 30° C.-50° C.-70° C. or 80° C. up to about 100° C.-115° C. or 120° C. The specific temperature depends on the type of solvent used in the reaction, and should be controlled to minimize excess side-product formation.

Although not to be bound by theory, Scheme 5, shows a proposed mechanism by which an example of a monotriflate isohexide can be prepared using a catalytic amount of a nucleophile and non-nucleophilic base.

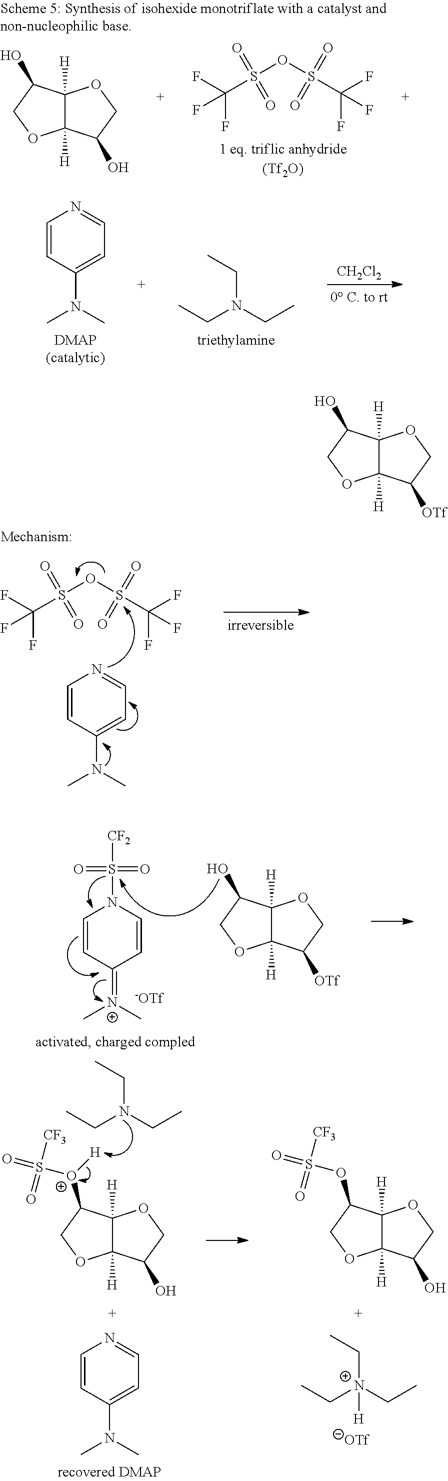

It is believed that the mechanism of this transformation is similar to that of the reaction in Scheme 2, but instead of using the liberated, nucleophilic base (pyridine), the reaction is performed with non-nucleophilic base (triethylamine) deprotonation.

In the second pathway, a combination of a non-nucleophilic base and a nucleophile is reacted. The non-nucleophilic base can be an amine, including but not limited to triethylamine, N,N-diisopropylethylamine (Hünig's base, (DIPEA or DIEA)), N-methylpyrrolidine, 4-methylmorpholine, and 1,4-diazabicyclo-(2,2,2)-octane (DABCO). In some embodiments, a tertiary amine base is combined with a nucleophilic catalyst, such as strongly nucleophilic 4-dimethylaminopyridine (DMAP). The nucleophile can be present in catalytic amounts, such as 1-5 mole % (0.01 to 0.05 equivalents) or less of the catalyst.

As a consideration in the execution of this second reaction pathway, one should control for the basicity of the reagents. This feature can affect the amounts of resulting elimination products (i.e., mono-unsaturated products). For example, an amine reagent generally will be strongly basic and will require more rigorously controlled conditions to minimize elimination products. The reaction would need to have narrower temperature and solvent parameters. For instance, at elevated temperatures the base-mediated elimination pathways are favored. Hence, the temperature would likely be held at a low temperature, such as 10° C. or 0° C. or below. In contrast, a thiol (e.g., cysteine) reagent (i.e., a non-basic nucleophile) gives rise to fewer elimination products. Hence, the non-basic reagent permits a relatively less stringent reaction environment (e.g., higher temperature) and allows for a reaction that can yield more of the desired product.

According to the present preparation, a triflate moiety attached to the isohexide activates a section of the molecule that can undergo facile substitution in a manner that cannot be efficiently accomplished without the presence of the triflate. The triflate imparts slightly elevated energy to the molecule. Any pathway that requires mono-substitution on the isohexide platform is greatly enhanced when the alcohol moiety is derivatized to the triflate moiety. Such substitution cannot occur without the presence of the triflate. While other leaving groups can be employed, such as tosylate and mesylate, these are much poorer nucleofuges than triflate, and often require elevated temperatures or more aggressive conditions which increases the likelihood of side reactions, such as particularly eliminations. This is one of the advantages that an isohexide monotriflate can afford for further synthesis of derivative compounds. In subsequent reactions to make derivative compounds, any number of nucleophilic substitutions can easily be effected, including but not limited to halides (I, Br, Cl), nitrogen centered (primary, secondary amines, azides, aromatic amines), carbon centered (Grignard, organolithiates, organocuprates) sulfur centered (thiols), and oxygen centered (alcohols, carboxylates). An example of this advantage is illustrated in Scheme 15A, in which an amine substitutes for the triflate moiety and then a long carbon chains attaches at the residual hydroxyl group.

A further point of interest is that the triflate, upon addition to the isohexide, effectuates in the isohexide a pronounced solvent solubility change, i.e., from being a hydrophilic (without the triflate) to being a hydrophobic compound. Thus, any risk for hydrolysis in the presence of water is reduced. More significantly, this modification can help with isolation of the monotriflate, for example, by means of liquid/liquid extraction from any unreacted original isohexide. In certain reactions, as little as about 1 equivalent or less of the triflate is added to the isohexide.

### B.—Monotriflates of the Isohexide Family

The isohexide family, because of their versatility that permits further chemical modifications, particularly isosorbide, is useful as a platform chemical. Compounds derived by further conversion of the isohexide monotriflate, for example, by etherification or esterification reactions, can serve as monomers and building blocks for new polymers and functional materials, new organic solvents, surfactants, for medical and pharmaceutical applications, and as fuels or fuel additives. (See. e.g., Marcus Rose et al., “Isosorbide as a Renewable Platform Chemical for Versatile Applications—Quo Vadis?,” CHEMSUSCHEM, vol. 5, pp. 167-176 (2012), contents incorporated herein by reference.)

One can synthesize monotriflate species from the three isohexide isomers equally well. The isohexide monotriflate isomers described herein present novel compositions of matter, which can be adapted to make valued building blocks to make chemical compounds for various applications, such as monomer units in polymers, dispersants, additives, lubricants, surfactants, and chiral auxiliaries.

When making derivative compounds, the monotriflate moiety may function either as an active site for nucleophilic substitution or as an inert moiety when derivatizing the other hydroxyl group of the isohexide molecule. Thus, by enhancing the chemical selectivity of reactive site toward nucleophilic substitution, the monotriflate serves as an electrophilic moiety that affords two distinct reactive sites on the isohexide, of particular use in the preparation of derivative compounds.

Isosorbide having both an endo and exo hydroxyl group, however, appears to be a more favored species for making the monotriflate species in terms of kinetics and control of reaction conditions. Generally, the three dimensional orientation of the hydroxyl groups has an impact on the rates at which the monotriflates are produced. In terms of the relative chemical reactive kinetics, endo positioned hydroxyl groups are more favored than exo positioned hydroxyl groups for the triflate derivatization. The ratio of endo:exo-oriented monotriflate species of isosorbide is about 2-3:1. Exo-oriented monotriflates exhibit enhanced reactivity during nucleophilic substitution. These characteristics will influence or dictate the nature of the chemical and physical properties of any resulting derivatized compounds.

Because of their underlying structural conformations, stereospecific transformation of isosorbide, isomannide, and isoidide generates four different isomers of isohexide mono-trifluoromethanesulfonates (i.e., monotriflates), as illustrated in Scheme 6.

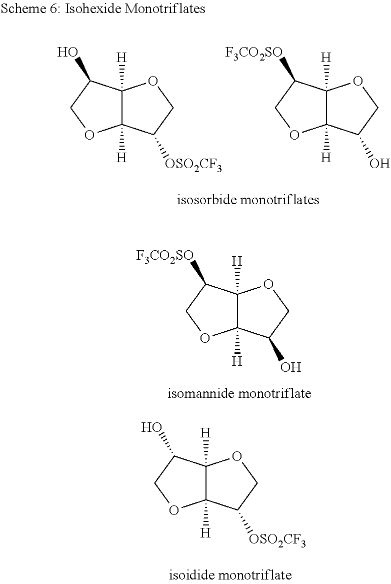

In another aspect, the present invention pertains to an isohexide monotriflate species and its use of as a platform chemical from which various different kinds of derivative compounds can be prepared. Table 1 lists the different isohexide monotriflate compounds that are prepared according to the an aspect of the present invention.

TABLE 1

| Common Name | IUPAC Name | Structure |
|---|---|---|
| Isosorbide monotriflate A | (3R,3aS,6S,6aR)-6- hydroxyhexahydrofuro[3,2- b]furan-3-yl trifluoromethanesulfonate | 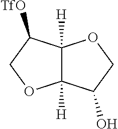 [SMILES: O=S(=O)(O[C@@H]1CO[C@H]2[C@@H]1OC[C@@H]2O)C(F)(F)F] |
| Isosorbide monotriflate B | (3S,3aS,6R,6aR)-6- hydroxyhexahydrofuro[3,2- b]furan-3-yl trifluoromethanesulfonate | 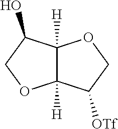 [SMILES: O=S(=O)(O[C@H]1CO[C@H]2[C@@H]1OC[C@H]2O)C(F)(F)F] |
| Isomannide monotriflate | (3R,3aS,6R,6aR)-6- hydroxyhexahydrofuro[3,2- b]furan-3-yl trifluoromethanesulfonate | 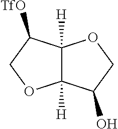 [SMILES: O=S(=O)(O[C@@H]1CO[C@H]2[C@@H]1OC[C@H]2O)C(F)(F)F] |
| Isoidide monotriflate | (3S,3aS,6S,6aR)-6- hydroxyhexahydrofuro[3,2- b]furan-3-yl trifluoromethanesulfonate | 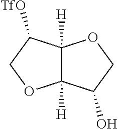 [SMILES: O=S(=O)(O[C@H]1CO[C@H]2[C@@H]1OC[C@@H]2O)C(F)(F)F] |
| | (3R,3aS,6aR)-2,3,3a,6a- tetrahydrofuro[3,2- b]furan-3-yl trifluoromethanesulfonate | 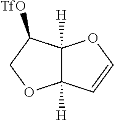 [SMILES: O=S(=O)(O[C@@H]1CO[C@@H]2C=CO[C@@H]21)C(F)(F)F] |
| | (3S,3aS,6aR)-2,3,3a,6a- tetrahydrofuro[3,2- b]furan-3-yl trifluoromethanesulfonate | 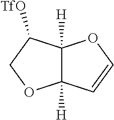 [SMILES: O=S(=O)(O[C@H]1CO[C@@H]2C=CO[C@H]12)C(F)(F)F] |

Given that the triflate moiety is one of the best nucleofuges, a variety of structurally distinct isohexide variants can be generated stereospecifically. A derivative compound can be prepared from one or more of the triflate (trifluoromethanesulfonate) compounds listed in Table 1, above. The manifold nucleophilic displacements are of particular interest in that they furnish Walden inversions of configurations of the isohexides, exemplified in Scheme 7 with the cyanation of isoidide monotriflate.

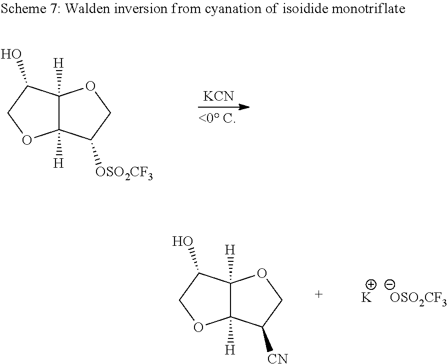

### C.—Derivative Compounds of Monotriflate Isoexhide

Once a monotriflate species is prepared according to an embodiment of the present invention, one may then produce various derivative compounds. In general, the process for making a derivative compound involves reacting an isohexide monotriflate species with at least, for example, an alcohol, aldehyde, amide, amine, inside, imine, carboxylic acid, cyanide, ester, ether, halide, thiol or other chemical groups. The derivative compound may include an organic moiety, for example, one or more of the following R-groups: an amide, amine, carboxylic acid, cyanide, ester, ether, thiol, alkane, alkene, alkyne, cyclic, aromatic, or nucleophilic moiety. Depending on the desired chemical or physical properties, one can select the monotriflate species having stereospecific conformations to modify in subsequent reactions to make derivative compounds that have different chemical and physical properties.

After derivatizing one of the hydroxyl groups with inflate moiety, one can react the remaining hydroxyl group on the isoxhexide, such as exemplified in Scheme 8 with α-bromoacetophenone.

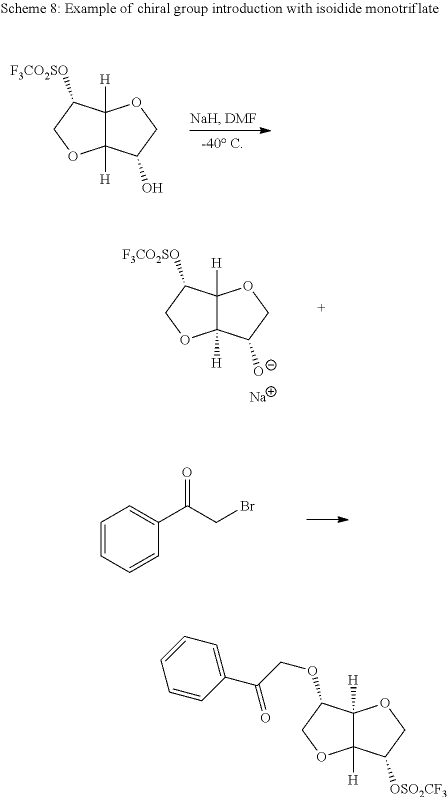

In other examples, the shielded, rigid orientation of the alcohol moiety necessitates nucleophilic addition/displacement reactions with the isohexide monotriflates to introduce valuable chirality to chemical platforms. Examples of such a reaction are presented in Schema 10, 11, 12, 15A and 15B.

### 1. Isosorbide Monotriflates:

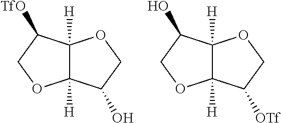

As mentioned before, monotriflates of isosorbide exhibit endo/exo orientations with respect to the triflate and alcohol moieties. This stereospecific arrangement allows for relatively unencumbered displacement of the triflate moiety with a nucleophile, such as butanethiol, and the respective generation of (exo thiol/exo hydroxy) isoidide and (endo thiol/endo hydroxy) isomannide derivatives. These diastereomers will manifest different physical and chemical properties from one another, such as melting and boiling points, phases, and reactivities. Scheme 9 shows an example of this reaction.

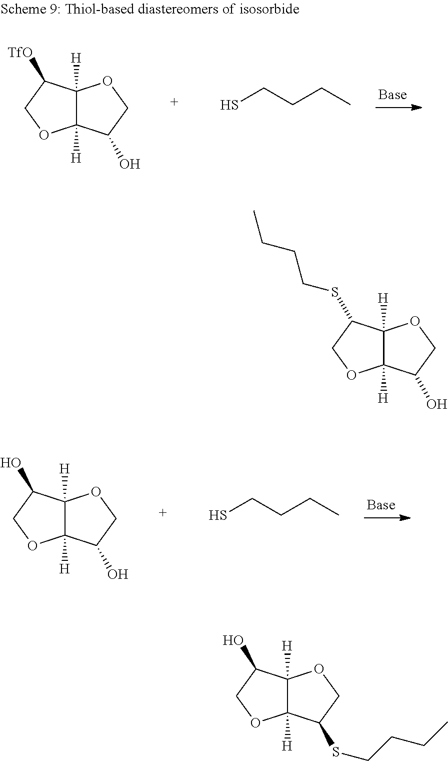

Functional conversion of the alcohol to an ester with butanoic acid, for example, preserves the (exo/endo) isosorbide platform, as shown in Scheme 10.

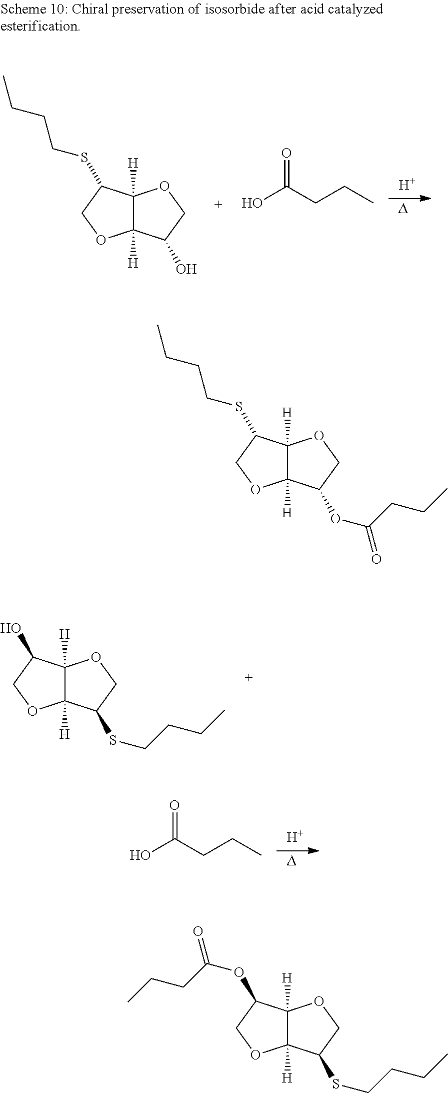

### 2. Isomannide Monotriflate

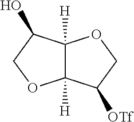 [SMILES: O=S(=O)(O[C@@H]1CO[C@H]2[C@@H]1OC[C@H]2O)C(F)(F)F]

Similarly, in a reaction using isomannide monotriflate, the stereospecific nucleophilic substitution of the triflate moiety with butanethiol, for example, engenders the (exo thiol/endo hydroxyl) isosorbide core through a Walden inversion, as shown in Scheme 11.

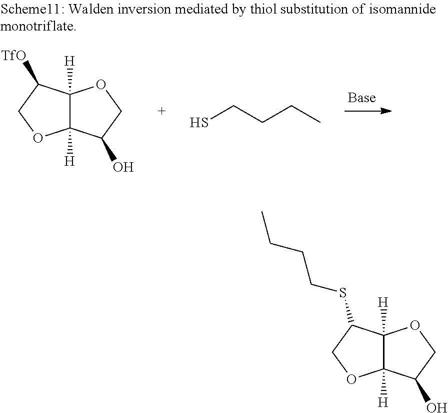

Further derivitization of the alcohol moiety, such as esterification with butanoic acid, maintains the (exo/exo) isoidide and (endo/endo) isomanide cores, as depicted in Scheme 12.

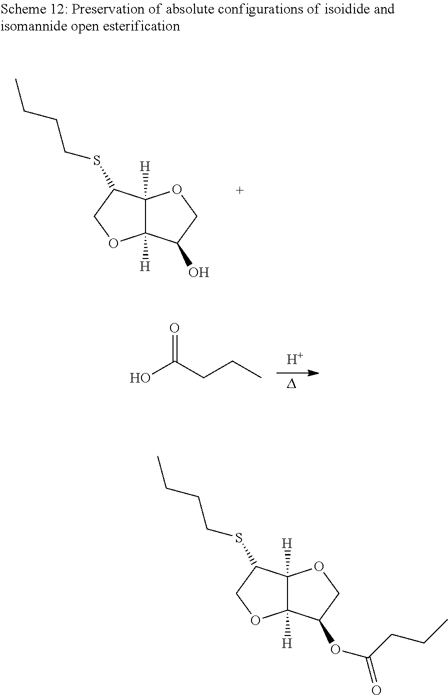

### 3. Isoidide Monotriflate

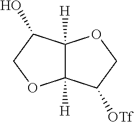 [SMILES: O=S(=O)(O[C@H]1CO[C@H]2[C@@H]1OC[C@@H]2O)C(F)(F)F]

When reacting isoidide monotriflate, the stereospecific nuceophilic substitution of the triflate moiety with butanethiol, for example, produces the (endo thiol/exo hydroxyl) isosorbide backbone, which exhibits entirely discrete physical and chemical properties than the aforementioned (endo hydroxyl/exo thiol) isosorbide diastereomer, as illustrated in Scheme 13.

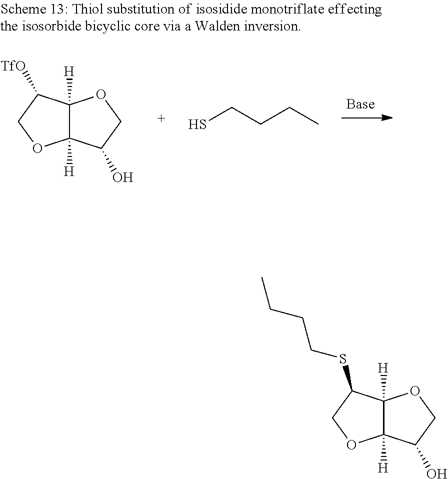

Esterification of the alcohol moiety with butanoic acid, for example, preserves the (endo/exo) isosorbide core, as depicted in Scheme 14.

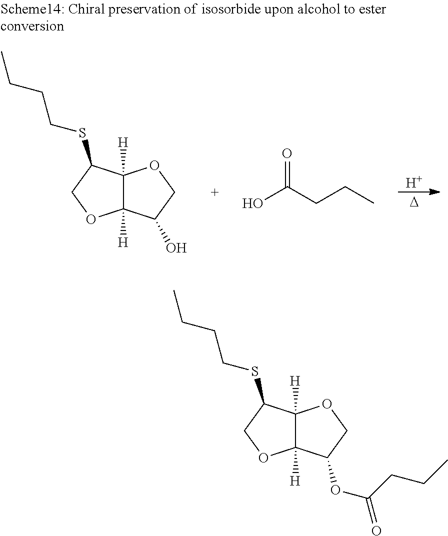

An example of a group of useful compounds that can be prepared from the monotriflates includes isohexide derived amphiphiles (i.e., a molecule having a water-soluble or hydrophilic polar moiety and a hydrophobic organic moiety). These compounds manifest discrete hydrophilic and hydrophobic zones that afford unique inter and intramolecular self-assemblies in response to environmental stimuli. Isohexide-based amphiphilic esters are predisposed to hydrolyze, particularly in commonly employed, non-neutral aqueous matrices. An alternative domain that wields a much greater robustness to hydrolytic conditions consists of alkyl ethers.

The difference in orientation between the functional groups on a monotriflate isohexide imparts unique amphiphilic properties to the corresponding mono ethers of the isohexides. Hence, an aspect of the present invention relates to the synthesis of a variety of either short (≦C₆), medium (C₇-C₁₂) or long (≧C₁₃) carbon chain isosorbide, isomannide and isoidide monoalkyl ethers. These scaffolds present attractive antecedents to different amphiphiles with potential uses, for instance, as surfactants, hydrophiles (e.g., carbon chain C₄-C₈), organogels, rheology adjustors, dispersants, emulsifiers, lubricants, plasticizers, chiral auxiliary compound with specific stereochemistry, among others.

In derivatizing the monotriflate species one can react, for example, an unhindered amine, a mono-amine, or including primary, secondary, and tertiary amines, such as with C₁-C₇, C₈-C₁₆, or C₁₇-C₂₅. For example, short chain (e.g., C₁-C₆) amines can be useful in making polymers, rheology adjustor compounds, plasticizers, and longer chain (e.g., C₈ or C₉-C₂₆) amines can be useful in preparing surfactants. The amine may include, for example, primary amines such as methylamine, ethylamine, propylamine, butylamine, isopropylamine, isobutylamine; or secondary amines, such as dimethylamine, diethylamine, diisopropylamine, diisobutylamine; or either primary and secondary species having a carbon chain up to icosan-1-amine (C₂₉).

One may subsequently modify the amine to generate an amine-based amphiphile with potential surfactant properties or other compounds manifesting useful commercial properties. (See e.g., J. Wu et al., “An Investigation of Polyamides Based in Isoidide-2,5-dimethyleneamine as a Green Rigid Building Block with Enhanced Reactivity,” MACROMOLECULES, vol. 45, pp. 9333-9346 (2012), incorporated by reference.)

An example of the preparation of an amine is illustrated in Scheme 15A. The derivative compound is an amphiphile, such as 2-(2-(2-(((3R,3aS,6S,6aR)-6-(octylamino)hexahydrofuro[3,2-b]furan-3-yl)oxy)ethoxy)ethoxy)-ethanol.

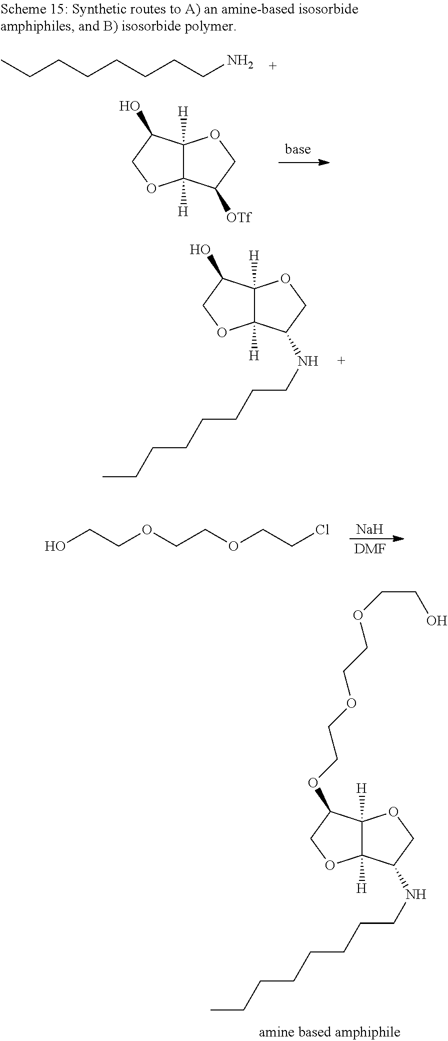

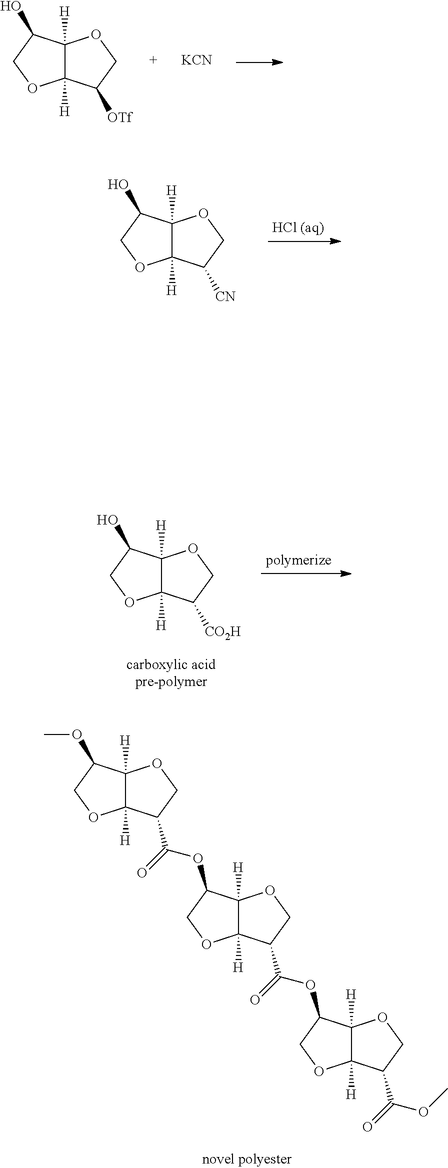

Alternatively, the derivative compound can be a monocarboxylic acid, such as at least one of: (3S,3aR,6R,6aR)-6-hydroxyhexahydrofuro[3,2-b]furan-3-carboxylic acid; or (3R,3aR,6S,6aR)-6-hydroxyhexahydrofuro[3,2-b]furan-3-carboxylic acid. The monocarboxylic acid can be subsequently polymerized, such as shown in Scheme 15B.

### Section II

### EXAMPLES

The present invention is further illustrated with reference to the following examples.

### Example 1

One can synthesize (3S,3aS,6S,6aR)-6-hydroxyhexahydrofuro[3,2-b]furan-3-yl-trifluoromethane-sulfonate. A (isoidide monotriflate), according to the following:

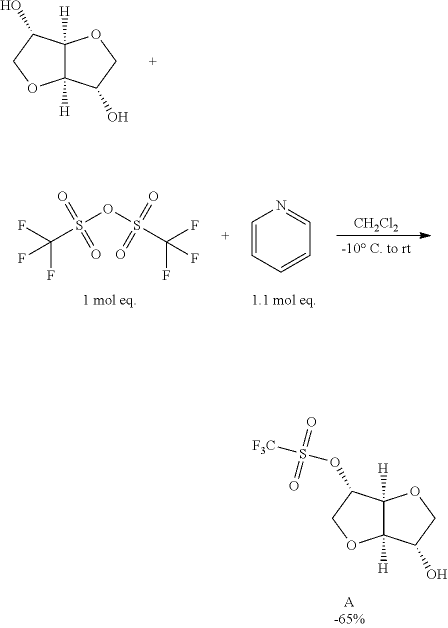

Experimental: Adapting a procedure as described in CHEMSUSCHEM, vol. 4, pp. 599-603, (2011), an oven-dried, 25 mL single neck round bottomed boiling flask, equipped with a ½″×⅜″ egg-shaped, PTFE-coated magnetic stir bar was charged with 409 mg of isoidide (2.80 mmol, 0.14M), 248 μL of pyridine, and 20 mL of methylene chloride. The neck was capped with a rubber septum and an argon inlet. With continued argon flow and vigorous stirring, the flask was immersed in an ice/brine bath (−10° C.) for approximately ˜10 minutes, and 470 μL of triflic anhydride (2.80 mmol) was added drop-wise over 15 minutes through the septum via syringe. The flask was removed from the ice bath after 30 minutes, warmed to room temperature, and reaction continued for another 30 more minutes. After this time, a profusion of solid was observed, suspended in a colorless solution.

In an alternate preparation protocol, an oven-dried, 25 mL single neck round bottomed boiling flask, equipped with a ½″×⅜″ egg-shaped, PTFE-coated magnetic stir bar was charged with 248 μL of pyridine and 20 mL of methylene chloride. The neck was capped with a rubber septum and an argon inlet was connected with 16′ needle. With continued argon flow and vigorous stirring, the flask was immersed in an ice/brine bath (−10° C.) for approximately ˜10 minutes, and 470 μL of triflic anhydride (2.80 mmol) added drop-wise over 15 minutes through the septum via syringe. Subsequently, 409 mg of isoidide (2.80 mmol) previously dissolved in 10 mL of methylene chloride was added drop-wise via syringe, while the flask remained at low temperature and under argon. After introduction of the isoidide, the ice bath was removed and the reaction continued for another 30 minutes.

An aliquot was withdrawn, diluted with methanol, and injected on a gas chromatography/mass spectrum analyzer (GC/MS) for compositional analysis. Two salient signals were observed. A first signal manifested a retention time of 12.90 minutes, m/z 260.0, consistent with putative compound B. (Not to be bound by theory, it is posited that compound B emanates from pyridine-induced elimination of the ditriflate analog in the manner illustrated in Scheme 17.)

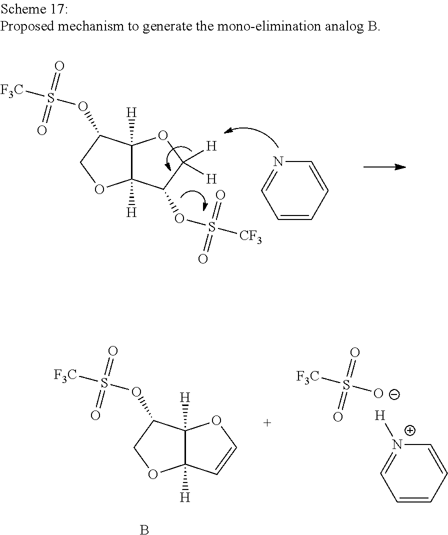

A second signal appeared at 13.06 minutes, m/z 278.0, congruent with the title compound A, indicating ˜65% molar yield.

Thin layer chromatography (TLC) was performed employing 1:1 hexanes:ethyl acetate as the mobile phase. Three distinct bands (cerium molybdate stain) were elicited; one evinced an rf of 0.85 (near solvent front), likely disclosing the elimination product B; one manifest an rf 0.38, consistent with target A; lastly, a dim band at the baseline was observed, indicative of residual isoidide. (The wide rf disparities would permit facile sequestration of the products by deploying flash silica gel chromatography.) The order of addition reagents appears not to be determinative of the reaction yield.

### Example 2

Synthesis of (3S,3aS,6R,6aR)-6-hydroxyhexahydrofuro[3,2-b]furan-3-trifluoromethane-sulfonate A and isomer (3S,3aR,6R,6aS)-6-hydroxyhexahydrofuro[3,2-b]furan-3-yl-trifluoromethane-sulfonate B (isosorbide monotriflate).

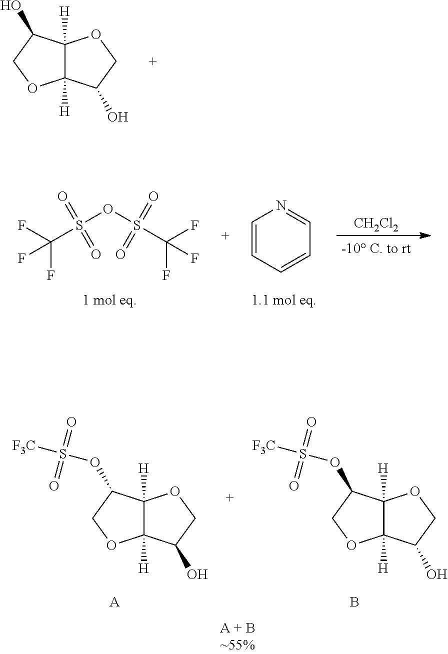

Experimental: An oven-dried, 25 mL single neck round bottomed boiling flask, equipped with a ½″×⅜″ egg-shaped, FTFE-coated magnetic stir bar was charged with 415 mg of isosorbide (2.84 mmol, 0.19M), 252 μL of pyridine (3.12 mmol), and 15 mL of methylene chloride. The neck was capped with a rubber septum and an argon inlet. With continued argon flow and vigorous stirring, the flask was immersed in an ice/brine bath (−10° C.) for approximately ˜10 minutes, and 477 μL of triflic anhydride (2.84 mmol) added dropwise over 15 minutes through the septum via syringe. The flask was removed from the ice bath after 30 minutes, warmed to room temperature, and reaction continued for 30 more minutes. After this time, a profusion of solid was observed, suspended in a light yellow solution. An aliquot was withdrawn, diluted with methanol, and injected on a GC/MS for compositional analysis. Three prominent signals were patent: 1) The first displayed a retention time of 12.29 minutes, m/z 278.0, consistent with title compounds A or B. 2) The second was revealed at 13.55 minutes, m/z 278.0, accordant with one of the title compounds A or B. These two signals combined to afford ˜55% molar yield for the reaction. An intense signal was disclosed at 13.72 minutes, m/z 260.0, denoting, perhaps, the aforementioned mono-unsaturated analog. Thin layer chromatography (TLC) was performed employing 1:1 hexanes:ethyl acetate as the mobile phase. Three distinct bands (cerium molybdate stain) were observed: one showed an rf of 0.88 (near solvent front) consistent the elimination compound highlighted in Scheme 1; one manifest an rf 0.39, consistent with overlapped A and B; lastly, a dim band at the baseline was descried, indicative of residual isosorbide.

### Example 3

Synthesis of (3R,3aS,6R,6aR)-6-hydroxyhexahydrofuro[3,2-b]furan-3-yl-trifluoromethane-sulfonate, A (isomannide monotriflate)

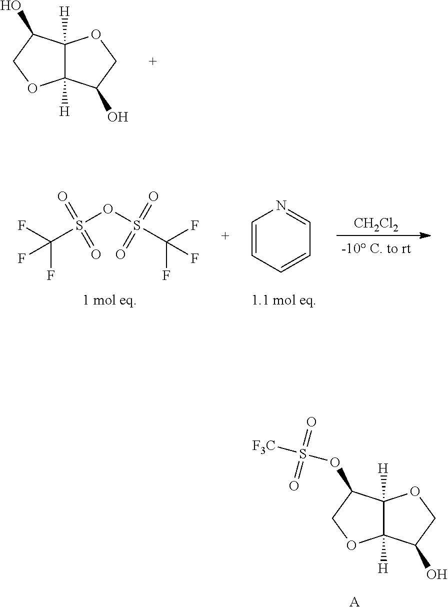

Experimental: An oven-dried, 25 mL single neck round bottomed boiling flask, equipped with a ½″×⅜″ egg-shaped, PTFE-coated magnetic stir bar was charged with 348 mg of isosorbide (2.38 mmol, 0.16M), 209 μL of pyridine (2.62 mmol), and 15 mL of methylene chloride. The neck was capped with a rubber septum and an argon inlet was connected with 16′ needle. With continued argon flow and vigorous stirring, the flask was immersed in an ice/brine bath (−10° C.) for approximately ˜10 minutes, then 400 μL of triflic anhydride (2.38 mmol) added dropwise over 15 minutes through the septum via syringe. The flask was removed from the ice bath after 30 minutes, warmed to room temperature, and reaction continued for 30 more minutes. After this time, a profusion of solid was observed, suspended in a colorless solution. An aliquot was withdrawn, diluted with methanol, and injected on a GC/MS for compositional analysis. Two striking signals were manifest: 1) The first displayed a retention time of 13.06 minutes, m/z 278.0, consistent with title compound A, and comprising a 51% molar yield for the reaction. 2) The second divulged a retention time of 14.38, m/z of 260.0, congruent with the previously mentioned mono-unsaturated compound. Three distinct bands (cerium molybdate stain) were observed; one displayed an rf of 0.81 (near solvent front) consistent with the elimination compound highlighted in Scheme 1; one manifest an rf 0.37, consistent with target A; and lastly a dim band at the baseline was espied indicative of residual isomannide. Pronounced discrepancies in TLC rf values of compounds in the crude matrix suggest that the individual isolation of the products, particularly the title compounds of the examples herein could be easily effected with the employ of flash silica gel chromatography. Furthermore, in instances where the aforementioned reactions were performed on larger scales, it is posited that short path pot distillation under vacuum would be efficacious in isolating individual products.

### Example 4

Synthesis of Amphiphilic 2-(2-(2-(((3S,3aS,6S,6aR)-6-(decylamino)hexahydrofuro[3,2-b]furan-3-yl)oxy)ethoxy)ethoxy)ethanol, from Isosorbide Triflate

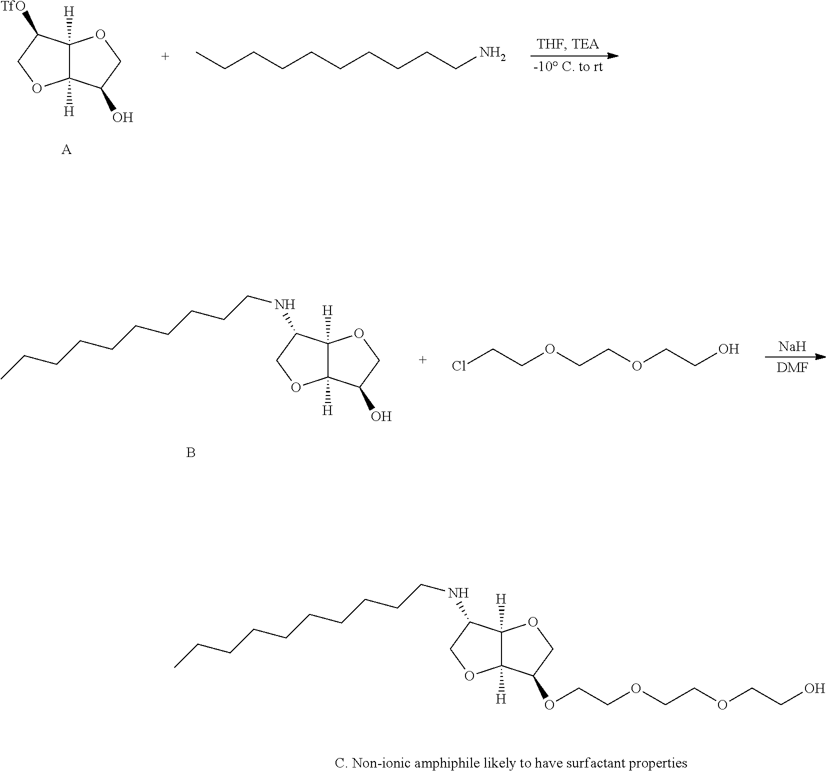

Experimental: Part 1, amino alcohol B. A septum capped 100 mL two neck round bottomed flask equipped with a magnetic stir bar and an argon inlet was charged with 2.00 g of isomannide monotriflate (7.19 mmol), 1.00 mL of triethylamine and 25 mL of anhydrous THF. The homogeneous mixture was then cooled to −10° C. in a saturated brine/ice bath. While stirring and under argon, 1.46 mL of decylamine (7.19 mmol), was added dropwise over 15 minutes. After complete addition, the ice bath was removed and reaction continued for another 2 h at room temperature. After this time, solids were filtered, excess solvent evaporated, and the brown, semisolid residue taken up in a minimum amount of methylene chloride and charged to a prefabricated flash column containing activated Brockmann basic alumina packing. The target amino alcohol B eluted with observed to elute with a 10:1 ethyl acetate/methanol solvent ratio as a 1.11 g of a light brown solid (54%). Spectroscopic elucidation with ¹H and ¹³C NMR and HRMS ensued, corroborating the high purity of B.

Part 2, nonanionic amphiphile C. A septum stoppered, two neck, 100 mL round bottomed flask outfitted with a magnetic stir bar and an argon gas inlet was charged with 1.40 g of the amino alcohol B (4.91 mmol), 196 mg of NaH (60% in mineral oil), and 25 mL of dry DMF. The solution was stirred for 15 minutes under an argon blanket, then 713 mL of 2-(2-(2-chloroethoxy)ethoxy)ethanol added dropwise via syringe. The reaction was continued overnight, after which time significant precipitate was observed. The solids were filtered and excess DMF removed by vacuum distillation, furnishing a light brown semi-solid matrix. This was taken up in a minimum amount of methylene chloride and charged to a prefabricated flash column packed with Brockmann activated basic alumina resin. The amphophilic compound C was observed to elute with a 6:1 ethyl acetate/methanol solvent composition, and, after concentration, appeared as a light brown semi-solid, 1.17 g (57%). Spectroscopic validation consisted of ¹H and ¹³C NMR and HRMS.

### Example 5

In the preparation of monocarboxylic acids, a three step process is employed. In the present example, (3S,3aR,6R,6aR)-6-hydroxyhexahydrofuro[3,2-b]furan-3-carboxylic acid (isosorbide monocarboxylic acid isomer D₁) is synthesized as follows:

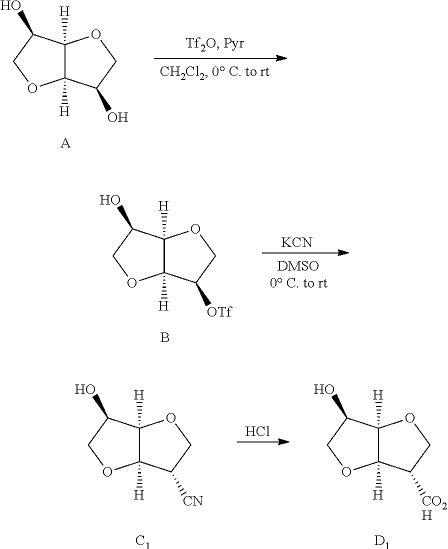

Step 1. Synthesis of (3R,3aS,6R,6aR)-6-hydroxyhexahydrofuro[3,2-b]furan-3-yl-trifluoromethane-sulfonate, B (isomannide monotriflate)

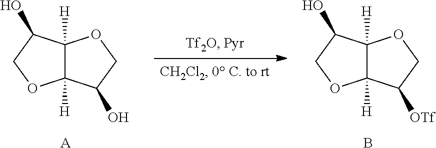

Experimental: An oven-dried, 100 mL single neck round bottomed boiling flask, equipped with a ½″×⅜″ egg-shaped, PTFE-coated magnetic stir bar was charged with 2.00 g of isomannide (13.68 mmol), 1.20 mL of dry pyridine (14.3 mmol), and 50 mL of methylene chloride. The neck was capped with a rubber septum and an argon inlet was connected via a 16′ needle. With continued argon flow and vigorous stirring, the flask was immersed in an ice/brine hath (−10° C.) for approximately ˜10 minutes, then 2.30 mL of triflic anhydride (13.04 mmol) added dropwise over 15 minutes through, the septum via syringe. The flask was removed from the ice hath after 30 minutes, warmed to room temperature, and reaction continued for overnight. After this time, a profusion of solid was observed, suspended in a colorless solution. The solids were filtered and filtrate decocted under vacuum, affording a colorless, viscous oil. This material was dissolved in a minimal amount of methylene chloride, adsorbed on silica gel (230-400 mesh, 40-63 μm) and charged to a prefabricated silica gel column, where flash chromatography with an effluent comprised of hexanes/ethyl acetate (5:1 to 1:1.5) furnished 2.05 g isomannide monotriflate as a white solid (53.8% theoretical). GC/MS (EI) analysis revealed a lone signal with retention time of 13.06 minutes, m/z 278.0, consistent with the monocarboxylic acid compound. ¹H NMR (CDCl₃, 400 MHz), δ (ppm) 5.69 (m, 1H), 4.24 (dd, J=7.2 Hz, J=5.6 Hz, 1H), 4.18 (dd, J=8.2 Hz, J=1.8 Hz, 2H), 4.08 (dd, J=8.4 Hz, J=1.6 Hz, 2H), 4.00 (dd, J=6.0 Hz, J=4.2 Hz, 1H), 3.86 (dd,J=8.2 Hz, J=6.0 Hz, 1H).
Step 2. Synthesis of (3S,3aR,6R,6aR)-6-hydroxyhexahydrofuro-[3,2-b]furan-3-carbonitrile (isosorbide mononitrile isomer C₁)

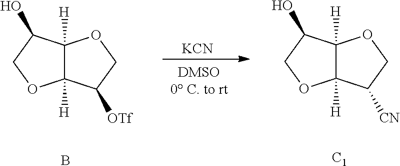

Experimental: A flame-dried, 100 mL round bottomed flask equipped with a ½″ PTFE-coated magnetic stir bar was charged with 468 mg of potassium cyanide (7.19 mmol) and 10 mL of anhydrous DMSO. The neck was capped with a rubber septum and argon inlet via 16′ needle and the flask subsequently immersed in a saturated brine/ice bath (˜10° C.). While stirring, 2.00 g of isomannide monotriflate B (7.19 mmol), previously dissolved in 10 mL of anhydrous DMSO, was added dropwise over a 30 minutes period. During the time of addition, the bath temperature was maintained at a constant −10° C. Afterwards, the ice bath was removed, matrix temperature gradually warmed to room temperature, and the reaction continued overnight. After this time, a dark solution was observed. Liquid-liquid extraction with a 100 mL volume of 1:1 water/methylene chloride effectively partitioned the isosorbide mononitrile isomer C₁ compound, and, after water layer with an additional 25 mL volume of methylene chloride, the combining of organic phases, and inspissation under vacuum, a dark, viscous residue was observed. This was dissolved in a minimal amount of methylene chloride, adsorbed in silica gel (230-400 mesh, 40-63 μm) and charged to a prefabricated column. Flash chromatography using an eluent comprised of hexanes/ethyl acetate (5:1 to 1:2) provided isosorbide mononitrile isomer C₁ as a light brown solid after concentration, 482 mg (43.4%). GC/MS (EI) analysis revealed a lone signal with retention time of 9.77 minutes, m/z 155.1. ¹H NMR (CDCl₃, 400 MHz), δ (ppm) 4.82 (m, 1H), 4.22 (dd, J=7.0 Hz, 5.2 Hz, 1H), 4.13 (dd, J=7.6 Hz, J=1.6 Hz, 2H), 4.01 (dd, J=8.0 Hz, J=2.2 Hz, 2H), 3.99 (dd, J=5.8 Hz, J=4.0 Hz, 1H), 3.87 (dd, J=8.4 Hz, J=6.0 Hz, 1H).
Step 3. Synthesis of (3S,3aR,6R,6aR)-6-hydroxyhexahydrofuro[3,2-b]furan-3-carboxylic acid (isosorbide monocarboxylic acid isomer D₁)

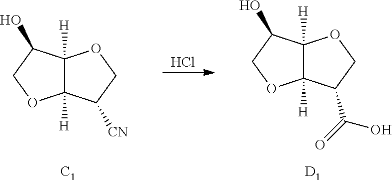

Experimental: A 25 mL round bottomed flask was charged with 300 mg of the isosorbide mononitrile isomer C₁ (1.9 mmol) and 5 mL of concentrated hydrochloric acid (about 12 M). The resulting suspension was then stirred at 75° C. under argon for 2 hours. After this time, the orange/red solution was cooled to room temperature, then concentrated using a short path condenser under reduced pressure (10 torr) and with gentle heating (50° C.). A dark brown precipitate was observed after overnight drying, weighing 330 mg (98%), and this was determined to be the title compound, isosorbide monocarboxylic acid isomer D₁, via spectroscopic analysis. ¹H NMR (D₂O, 400 MHz) δ 4.92 (m, 2H), 4.08 (m, 2H), 3.92 (m, 2H), 3.18 (m, 2H); HRMS (M+) Predicted for C₇H₁₆O₆: 174.1513; Found. 174.1502.

### Example 6

A three-step preparation of a monocarbocylic acid using isoidide, (3R,3aR,6S,6aR)-6-hydroxyhexahydroxyfuro[3,2-b]furan-3-carboxylic acid (isosorbide monocarboxylic acid isomer D₂), is as follows:

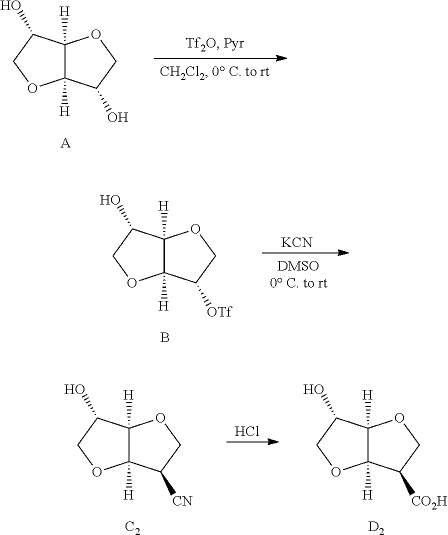

Step 1. Synthesis of (3S,3aS,6S,6aR)-6-hydroxyhexahydroxyfuro[3,2-b]furan-3-yl-trifluoromethane-sulfonate B (isoidide monotriflate)

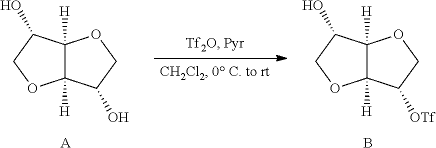

Experimental: An oven-dried, 100 mL single neck round bottomed boiling flask, equipped with a ½″×⅜″ egg-shaped, PTFE-coated magnetic stir bar was charged with 2.00 g of isoidide (13.68 mmol), 1.20 mL of dry pyridine (14.3 mmol), and 50 mL of methylene chloride. The neck was capped with a rubber septum and an argon inlet was connected via a 16′ needle. With continued argon flow and vigorous stirring, the flask was immersed in an ice/brine bath (−10° C.) for approximately ˜10 minutes, then 2.30 mL of triflic anhydride (13.04 mmol) added dropwise over 15 minutes through the septum via syringe. The flask was removed from the ice bath after 30 minutes, warmed to room temperature, and reaction continued for overnight. After this time, a profusion of solid was observed, suspended in a colorless solution. The solids were filtered and filtrate decocted under vacuum, affording a colorless, viscous oil. This material was dissolved in a minimal amount of methylene chloride, adsorbed on silica gel (230-400 mesh, 40-63 μm) and charged to a prefabricated silica gel column, where flash chromatography with an effluent comprised of hexanes/ethyl acetate (2:1 to 1:1.5) furnished 2.16 g isoidide monotriflate as a white solid (56.7% theoretical). GC/MS (EI) analysis revealed a lone signal with retention time of 12.90 minutes, m/z 260.0, consistent with the title compound.
Step 2. Synthesis of (3R,3aR,6S,6aR)-6-hydroxyhexahydroxyfuro[3,2-b]furan-3-carbonitrile (isosorbide mononitrile isomer C₂)

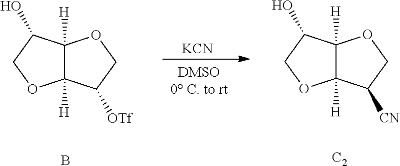

Experimental: A flame-dried, 100 mL round bottomed flask equipped with a ½″ PTFE-coated magnetic stir bar was charged with 468 mg of potassium cyanide (7.19 mmol) and 10 mL of anhydrous DMSO. The neck was capped with a rubber septum and argon inlet via 16′ needle and the flask subsequently immersed in a saturated brine/ice bath (˜10° C.). While stirring, 2.00 g of isoidide monotriflate B (7.19 mmol), previously dissolved in 10 mL of anhydrous DMSO, was added dropwise over a 30 minutes period. During the time of addition, the bath temperature was maintained at a constant −10° C. Afterwards, the ice bath was removed, matrix temperature gradually warmed to room temperature, and the reaction continued overnight. After this time, a dark solution was observed. Liquid-liquid extraction with a 100 mL volume of 1:1 water/methylene chloride effectively partitioned the title compound, isosorbide mononitrile isomer C₂, and after water layer with an additional 25 mL volume of methylene chloride, the combining of organic phases, and concentration under vacuum, a light brown, viscous residue was observed. This was dissolved in a minimal amount of methylene chloride, adsorbed in silica gel (230-400 mesh, 40-63 μm) and charged to a prefabricated column. Flash chromatography using an eluent comprised of hexanes/ethyl acetate (2:1 to 1:2) provided the title compound, isosorbide mononitrile isomer C₂, as an off-white solid after concentration, 513 mg 46.2%). GC/MS (EI) analysis revealed a lone signal with retention time of 9.54 minutes, m/z 155.1.
Step 3. Synthesis of (3R,3aR,6S,6aR)-6-hydroxyhexahydrofuro[3,2-b]furan-3-carboxylic acid, isosorbide monocarboxylic acid isomer D₂

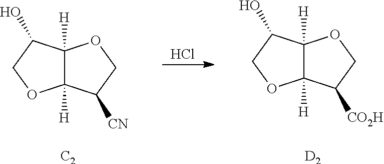

Experimental: A 25 mL round bottomed flask was charged with 300 mg of the isosorbide mononitrile isomer C₂ (1.9 mmol) and 5 mL of concentrated hydrochloric acid (about 12 M). The resulting suspension was then stirred at 75° C. under argon for 2 hours. After this time, the orange/red solution was cooled to room temperature, and then concentrated using a short path condenser under reduced pressure (10 torr) and with gentle heating (50° C.). A dark brown precipitate was observed after overnight drying, weighing 318 mg (94%), and this was determined to be the title compound, isosorbide monocarboxylic acid isomer D₂, via nuclear magnetic resonance spectroscopy; ¹H NMR (D₂O, 400 MHz) δ (ppm) 4.97 (m, 2H), 4.04 (m, 2H), 3.87 (m, 2H), 3.16 (m, 2H). ¹³C NMR (D₂O, 400 MHz) δ 177.3, 93.1, 87.5, 70.4, 67.4, 62.2, 56.1.

Although the present invention has been described generally and by way of examples, it is understood by those persons skilled in the art that the invention is not necessarily limited to the embodiments specifically disclosed, and that modifications and variations can be made without departing from the spirit and scope of the invention. Thus, unless changes otherwise depart from the scope of the invention as defined by the following claims, they should be construed as included herein.

We claim:

1. A process of preparing an isohexide monotriflate, comprising: reacting a mixture or an isohexide, a trifluoromethanesulfonate anhydride, and a reagent of either 1) a nucleophilic base or 2) a combination of a non-nucleophilic base and a nucleophile.

2. The process according to claim 1, wherein said isohexide is at least one of the following: isosorbide, isomannide, and isoidide.

3. The process according to claim 1, wherein said nucleophilic base is at least one of: pyridine, dimethyl-aminopyridine, imidazole, pyrrolidine, and morpholine.

4. The process according to claim 1, wherein said non-nucleophilic base is an amine selected from the group consisting of: triethylamine, Hünig's base (N,N-diisopropylethylamine), N-methylpyrrolidine, 4-methylmorpholine, and 1,4-diazabicyclo-(2,2,2)-octane (DABCO).

5. The process according to claim 1, wherein said nucleophile is 4-dimethylaminopyridine (DMAP).

6. The process according to claim 1, wherein when said reagent is a nucleophilic base, said reaction is conducted at an initial temperature of about 1° C. or less.

7. The process according to claim 6, wherein said initial temperature is in a range between about −5° C. and about −80° C.

8. The process according to claim 6, wherein said process involves reacting said trifluoromethanesulfonate anhydride with said nucleophilic base at temperatures of 0° C. or below prior to an addition of said isohexide.

9. The process according to claim 1, wherein when said reagent is a combination of a non-nucleophilic base and a nucleophile, said reaction is conducted at about ambient room temperature or greater.

10. The process according to claim 1, wherein said process produces primarily isohexide mono-triflates in molar yields of at least 50% from said isohexide starting materials.

11. A chemical compound comprising an isohexide monotriflate selected from the group consisting of:
a) (3R,3aS,6S,6aR)-6-hydroxyhexahydrofuro[3,2-b]furan-3-yl trifluoromethanesulfonate, with a structure: 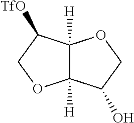 [SMILES: O=S(=O)(O[C@@H]1CO[C@H]2[C@@H]1OC[C@@H]2O)C(F)(F)F]
b) (3S,3aS,6R,6aR)-6-hydroxyhexahydrofuro[3,2-b]furan-3-yl trifluoromethanesulfonate, with a structure: 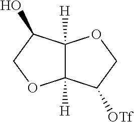 [SMILES: O=S(=O)(O[C@H]1CO[C@H]2[C@@H]1OC[C@H]2O)C(F)(F)F]
c) (3R,3aS,6R,6aR)-6-hydroxyhexahydrofuro[3,2-b]furan-3-yl trifluoromethanesulfonate, with a structure: 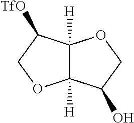 [SMILES: O=S(=O)(O[C@@H]1CO[C@H]2[C@@H]1OC[C@H]2O)C(F)(F)F]
d) (3S,3aS,6S,6aR)-6-hydroxyhexahydroxyfuro[3,2-b]furan-3-yl trifluoromethanesulfonate, with a structure: 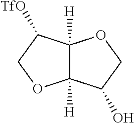 [SMILES: O=S(=O)(O[C@H]1CO[C@H]2[C@@H]1OC[C@@H]2O)C(F)(F)F]
e) (3R,3aS,6aR)-2,3,3a,6a-tetrahydrofuro[3,2-b]furan-3-yl trifluoromethanesulfonate, with a structure: 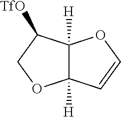 [SMILES: O=S(=O)(O[C@@H]1CO[C@@H]2C=CO[C@@H]21)C(F)(F)F]
f) (3S,3aS,6aR)-2,3,3a,6a-tetrahydrofuro[3,2-b]furan-3-yl trifluoromethanesulfonate, with a structure:

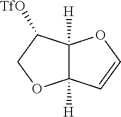 [SMILES: O=S(=O)(O[C@H]1CO[C@@H]2C=CO[C@H]12)C(F)(F)F]

12. A process for making a derivative compound of an isohexide monotriflate, comprising:
reacting an isohexide monotriflate species selected from the group consisting of:
a) (3R,3aS,6S,6aR)-6-hydroxyhexahydrofuro[3,2-b]furan-3-yl trifluoromethanesulfonate;
b) (3S,3aS,6R,6aR)-6-hydroxyhexahydrofuro[3,2-b]furan-3-yl trifluoromethanesulfonate;
c) (3R,3aS,6R,6aR)-6-hydroxyhexahydrofuro[3,2-b]furan-3-yl trifluoromethanesulfonate;
d) (3S,3aS,6S,6aR)-6-hydroxyhexahydrofuro[3,2-b]furan-3-yl trifluoromethanesulfonate;
e) (3R,3aS,6aR)-2,3,3a,6a-tetrahydrofuro[3,2-b]furan-3-yl trifluoromethanesulfonate;
f) (3S,3aS,6aR)-2,3,3a,6a-tetrahydrofuro[3,2-b]furan-3-yl trifluoromethanesulfonate,
with an at least one the following species: an alcohol, aldehyde, amide, amine, imide, imine, carboxylic acid, cyanide, ester, ether, halide, and thiol.

13. A derivative compound prepared front one or more of the following:
a) (3R,3aS,6S,6aR)-6-hydroxyhexahydrofuro[3,2-b]furan-3-yl trifluoromethanesulfonate;
b) (3S,3aS,6R,6aR)-6-hydroxyhexahydrofuro[3,2-b]furan-3-yl trifluoromethanesulfonate;
c) (3R,3aS,6R,6aR)-6-hydroxyhexahydrofuro[3,2-b]furan-3-yl trifluoromethanesulfonate;
d) (3S,3aS,6S,6aR)-6-hydroxyhexahydrofuro[3,2-b]furan-3-yl trifluoromethanesulfonate;
e) (3R,3aS,6aR)-2,3,3a,6a-tetrahydrofuro[3,2-b]furan-3-yl trifluoromethanesulfonate;
f) (3S,3aS,6aR)-2,3,3a,6a-tetrahydrofuro[3,2-b]furan-3-yl trifluoromethanesulfonate.

14. The derivative compound according to claim 13, wherein said derivative compound includes an R-group with at least one of the following; an amine, carboxylic acid, amide, ester, ether, thiol alkane, alkene, alkyne, cyclic, aromatic, or a nucleophilic moiety.

15. The derivative compound according to claim 14, wherein said derivative compound is a mono-amine.

16. The derivative compound according to claim 14, wherein said monoamine is selected from the group consisting of: C₁-C₂₅ primary, secondary, and tertiary amines.

17. The derivative compound according to claim 14, wherein said derivative compound is a monocarboxylic acid.

18. The derivative compound according to claim 17, wherein said derivative compound is at least one of: (3S,3aR,6R,6aR)-6-hydroxyhexahydrofuro[3,2-b]furan-3caboxylic acid; or (3R,3aR,6S,6aR)-6-hydroxyhexahydrofuro[3,2-b]furan-3-carboxylic acid.

19. The derivative compound according to claim 14, wherein said derivative compound is an amphiphilic.

20. The derivative compound according to claim 19, wherein said amphiphilic is: a surfactant, a hydrophile, an organogel, a rheology adjustor, a dispersant, or a plasticizer.

21. The derivative compound according to claim 19, wherein said amphiphile is a chiral auxiliary compound.

22. The derivative compound according to claim 14, wherein said derivative compound is a thiol or thiol-ether.

23. A derivative compound prepared from an isohexide monotriflate selected from the group consisting of:
a) (3R,3aS,6S,6aR)-6-hydroxyhexahydrofuro[3,2-b]furan-3-yl trifluoromethanesulfonate, with a structure: 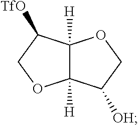 [SMILES: O=S(=O)(O[C@@H]1CO[C@H]2[C@@H]1OC[C@@H]2O)C(F)(F)F]
b) (3S,3aS,6R,6aR)-6-hydroxyhexahydrofuro[3,2-b]furan-3-yl trifluoromethanesulfonate, with a structure: 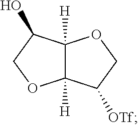 [SMILES: O=S(=O)(O[C@H]1CO[C@H]2[C@@H]1OC[C@H]2O)C(F)(F)F]
c) (3R,3aS,6R,6aR)-6-hydroxyhexahydroxyfuro[3,2-b]furan-3-yl trifluoromethanesulfonate, with a structure: 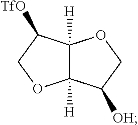 [SMILES: O=S(=O)(O[C@@H]1CO[C@H]2[C@@H]1OC[C@H]2O)C(F)(F)F]
d) (3S,3aS,6S,6aR)-6-hydroxyhexahydroxyfuro[3,2-b]furan-3-yl trifluoromethanesulfonate, with a structure: 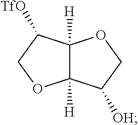 [SMILES: O=S(=O)(O[C@H]1CO[C@H]2[C@@H]1OC[C@@H]2O)C(F)(F)F]
e) (3R,3aS,6aR)-2,3,3a,6a-tetrahydroxyfuro[3,2-b]furan-3-yl trifluoromethanesulfonate, with a structure: 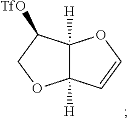 [SMILES: O=S(=O)(O[C@@H]1CO[C@@H]2C=CO[C@@H]21)C(F)(F)F]
f) (3S,3aS,6aR)-2,3,3a,6a-tetrahydroxyfuro[3,2-b]furan-3-yl trifluoromethanesulfonate, with a structure: 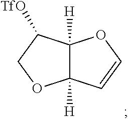 [SMILES: O=S(=O)(O[C@H]1CO[C@@H]2C=CO[C@H]12)C(F)(F)F]
said derivative compound having a general formula: X—R or R₁—X—R₂, wherein said X is said isohexide monotriflate as modified with R, R₁, R₂; and R, R₁, R₂ each is an organic moiety that contains at least one of the following: an amine, amide, carboxylic acid, cyanide, ester, ether, thiol, alkane, alkene, alkyne, cyclic, aromatic, or a nucleophilic moiety.

24. The derivative compound according to claim 23, wherein said derivative compound is at least one of the following: 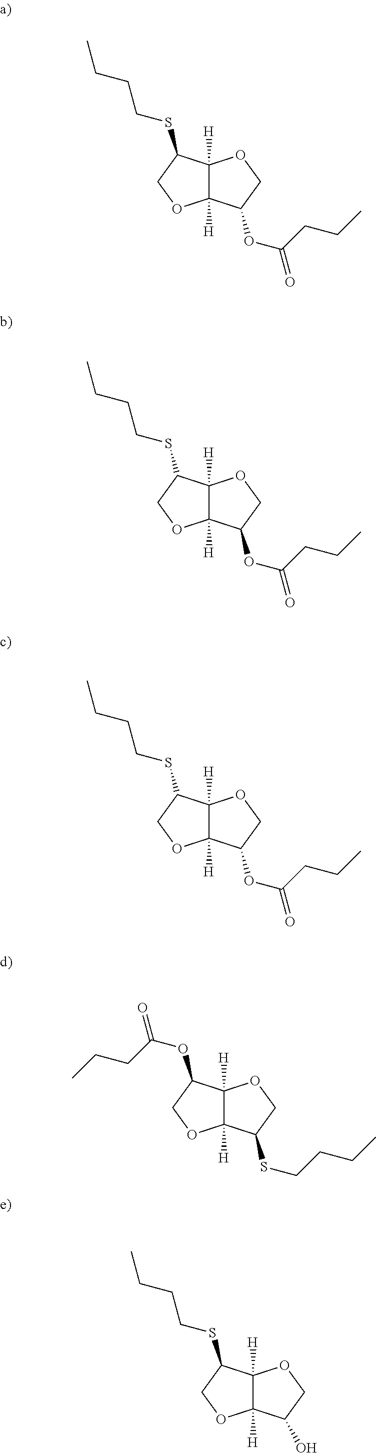 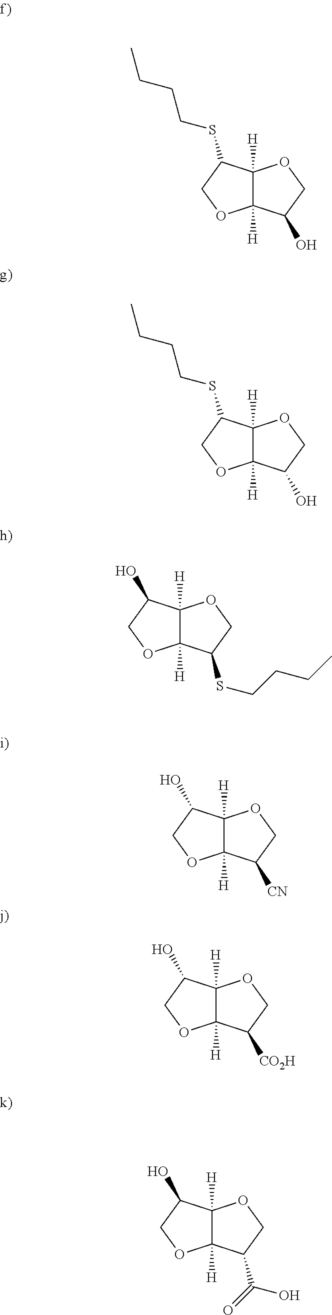 [chemical structure]
